(19) United States
(12) Patent Application Publication
(10) Pub. No.: US 2025/0162962 A1
(43) Pub. Date: May 22, 2025

(54) METHOD OF PRODUCING LIQUID HYDROCARBONS FROM A SYNGAS

(71) Applicant: JOHNSON MATTHEY DAVY TECHNOLOGIES LIMITED, London (GB)

(72) Inventors: Rob Miles BAKER, London (GB); Andrew James COE, London (GB)

(21) Appl. No.: 18/834,053

(22) PCT Filed: Mar. 16, 2023
(86) PCT No.: PCT/GB2023/050624

§ 371 Date: Jul. 29, 2024

(30) Foreign Application Priority Data
Apr. 1, 2022 (GB) 2204765.8

(51) Int. Cl. C07C 1/32; B01D 53/14; C01C 1/02

(52) U.S. Cl. CPC C07C 1/323; B01D 53/1493; C01C 1/026; B01D 2252/103; B01D 2257/406; C07C 2523/75

(57) ABSTRACT

A method of producing liquid hydrocarbons from a syngas, the method comprising: providing a first syngas containing hydrogen cyanide; converting at least a portion of the hydrogen cyanide in the first syngas to ammonia to provide a second syngas enriched in ammonia and depleted in hydrogen cyanide; passing the second syngas to a scrubber and contacting the second syngas with a scrubbing liquid, whereby at least a portion of ammonia contained in the second syngas is retained in the scrubbing liquid to form a third syngas depleted in ammonia and hydrogen cyanide; and passing the third syngas through a Fischer-Tropsch reaction chamber to produce a liquid hydrocarbon product, wherein passing the third syngas through a Fischer-Tropsch reaction chamber to produce a liquid hydrocarbon product comprises contacting the third syngas with a catalyst comprising a metal selected from cobalt, iron and ruthenium.

### FIELD OF THE INVENTION

The invention relates to a method of producing liquid hydrocarbons from a syngas.

### BACKGROUND OF THE INVENTION

The Fischer-Tropsch process is a collection of chemical reactions that converts a mixture of carbon monoxide and hydrogen into liquid hydrocarbons. These reactions occur in the presence of metal catalysts, typically at temperatures of 150-300° C. and pressures of one to several tens of atmospheres. The Fischer-Tropsch process involves a series of chemical reactions that produce a variety of hydrocarbons, ideally having the formula (CₙH₂ₙ₊₂). The more useful reactions produce alkanes as follows:

(2n+1)H₂+nCO→CₙH₂ₙ₊₂+nH₂O

where n is typically 1-100, or higher. The formation of methane (n=1) is unwanted. Most of the alkanes produced tend to be straight-chain, and are suitable to be upgraded to produce middle distillate fuels such as diesel and jet fuel. In addition to alkane formation, competing reactions give small amounts of alkenes, as well as alcohols and other oxygenated hydrocarbons. Co-produced water is a by-product, which is separated from the products of the Fischer-Tropsch reaction. The Fischer-Tropsch reaction is a highly exothermic reaction due to a standard reaction enthalpy (ΔH) of −165 KJ/mol CO combined.

Synthesis gas (syngas) feed to a Fischer-Tropsch unit can be derived from a number of feedstocks; for example, natural gas via steam reforming and/or auto-thermal reforming, municipal solid waste and biomass via high-temperature gasification or carbon dioxide and hydrogen via a reverse water-gas shift. The syngas produced by these processes typically contains ppm levels of hydrogen cyanide and ammonia, which deactivate the Fischer-Tropsch catalyst, and so ideally the hydrogen cyanide and ammonia are removed down to single-digit ppb levels. To remove these species from the syngas, the hydrogen cyanide is typically converted to ammonia via hydrolysis and then the ammonia removed using a wet scrubber. Achieving ppb levels of ammonia by conventional scrubbing is technically challenging.

U.S. Pat. No. 6,107,353 describes a method of removing hydrogen cyanide and ammonia from a syngas prior to a hydrocarbon synthesis reaction. The method involves passing the syngas to a catalyst to hydrolyse hydrogen cyanide to ammonia. The hydrolysed gas removed from the hydrolysis zone is then contacted with water in a scrubbing zone to dissolve ammonia. However, in order to achieve ppb levels of hydrogen cyanide and ammonia, the method requires the use of hydrogen-cyanide absorption zones prior to entry of the syngas into the Fischer-Tropsch reactor. The use of such hydrogen-cyanide absorption zones increases the complexity of the method.

The present invention seeks to tackle at least some of the problems associated with the prior art or at least to provide a commercially acceptable alternative solution thereto.

### SUMMARY OF THE INVENTION

One aspect of the present disclosure is directed to a method of producing liquid hydrocarbons from a syngas, the method comprising:
providing a first syngas containing hydrogen cyanide;
converting at least a portion of the hydrogen cyanide in the first syngas to ammonia to provide a second syngas enriched in ammonia and depleted in hydrogen cyanide;
passing the second syngas to a scrubber and contacting the second syngas with a scrubbing liquid, whereby at least a portion of ammonia contained in the second syngas is retained in the scrubbing liquid to form a third syngas depleted in ammonia and hydrogen cyanide; and
passing the third syngas through a Fischer-Tropsch reaction chamber to produce a liquid hydrocarbon product, wherein passing the third syngas through a Fischer-Tropsch reaction chamber to produce a liquid hydrocarbon product comprises contacting the third syngas with a catalyst comprising a metal selected from cobalt, iron and ruthenium;
characterised in that the scrubbing liquid comprises a co-produced water separated from products recovered from the Fischer-Tropsch reaction chamber.

### BRIEF DESCRIPTION OF THE DRAWINGS

FIG. 1 shows a flow diagram of an example of a method according to the present invention.

### DETAILED DESCRIPTION OF THE INVENTION

In a first aspect, the present disclosure is directed to a method of producing liquid hydrocarbons from a syngas, the method comprising:
providing a first syngas containing hydrogen cyanide;
converting at least a portion of the hydrogen cyanide in the first syngas to ammonia to provide a second syngas enriched in ammonia and depleted in hydrogen cyanide;
passing the second syngas to a scrubber and contacting the second syngas with a scrubbing liquid, whereby at least a portion of ammonia contained in the second syngas is retained in the scrubbing liquid to form a third syngas depleted in ammonia and hydrogen cyanide; and
passing the third syngas through a Fischer-Tropsch reaction chamber to produce a liquid hydrocarbon product, wherein passing the third syngas through a Fischer-Tropsch reaction chamber to produce a liquid hydrocarbon product comprises contacting the third syngas with a catalyst comprising a metal selected from cobalt, iron and ruthenium;
characterised in that the scrubbing liquid comprises co-produced water separated from products recovered from the Fischer-Tropsch reaction chamber.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

Advantageously, in contrast to conventional methods, the use of the co-produced water as the scrubbing liquid, which is performed as a final treatment of the syngas upstream of the Fischer-Tropsch reaction chamber, reduces the risk of catalyst poisoning that is present in conventional washing methods. The ammonia removal capacity of the scrubbing liquid is also improved by the presence of carbon dioxide dissolved in the co-produced water. If the water of the scrubbing liquid is not enriched with carbon dioxide, then more scrubbing stages or more scrubbing liquid may be required to reduce the ammonia levels. Accordingly, it is possible to reduce the ammonia and hydrogen cyanide contents of the syngas to single-digit ppb levels. As a result, poisoning of the Fischer-Tropsch catalyst is reduced, meaning that the method is more efficient due to the need to regenerate or replace the Fischer-Tropsch catalyst less regularly. The method uses the third syngas, without further treatment, as the feed to the Fischer-Tropsch reaction chamber. Accordingly, the present invention avoids the use of downstream hydrogen-cyanide and ammonia absorption beds, thereby resulting in a more simplified and cost-effective method in comparison to other methods.

The term “liquid hydrocarbons” as used herein may encompass species formed of carbon and hydrogen that are liquid at room temperature and pressure. The hydrocarbons typically comprise alkanes, and typically comprise from 5 to 100, or more, carbon atoms per molecule.

The term “syngas” or “synthesis gas” as used herein may encompass a gas mixture containing hydrogen and carbon monoxide. In the method of the present invention, the first syngas comprises carbon monoxide (i.e. CO), hydrogen (i.e. molecular hydrogen H₂), hydrogen cyanide (i.e. HCN) and typically less than 1% by volume carbon dioxide (CO₂). The syngas may comprise other gases such as, for example, water, methane, ammonia and sulphur-containing gas, e.g. hydrogen sulphide (i.e. H₂S), as well as solid species such as, for example, dust and coke. Syngas is typically produced from the gasification of a carbonaceous material. In some arrangements, providing the first syngas containing hydrogen cyanide may comprise the gasification of biomass, municipal waste or coal, the steam reforming of a hydrocarbon feedstock or recovering it from a reverse-water-gas shift unit. In the present invention, the first syngas is preferably non-fossil fuel based, i.e produced by the gasification of biomass and/or municipal waste. Whereas such feeds are more sustainable than fossil fuels, they do create additional problems with respect to catalyst poisoning. Alternatively, the first syngas may be recovered from a reverse-water-gas shift unit used to convert carbon dioxide, such as carbon dioxide separated from combustion gases, air or other chemical processes, into carbon monoxide with hydrogen. The components of the syngas will vary depending on its method of manufacture and the starting materials used.

The method involves passing the second syngas to a scrubber and contacting the second syngas with a scrubbing liquid. Scrubbers and scrubbing liquids are known in the art. Removal efficiency of ammonia may be improved by increasing residence time in the scrubber or by the increase of surface area of the scrubbing liquid by the use of, for example, trays, structured packing or random packing.

The third syngas is passed through a Fischer-Tropsch reaction chamber, which contains a Fischer-Tropsch catalyst. Fischer-Tropsch reaction chambers are known in the art.

The scrubbing liquid comprises a co-produced water. The co-produced water may be separated from the products recovered from the Fischer-Tropsch reaction chamber using conventional separation equipment.

The co-produced water preferably comprises at least 0.01 mol/L carbon dioxide, more preferably at least 0.02 mol/L carbon dioxide. Such concentrations of carbon dioxide may result in a particularly high ammonia removal capacity of the scrubbing liquid.

In a preferred embodiment, the co-produced water is preferably saturated with carbon dioxide under the temperature and pressure conditions of the scrubber.

The liquid hydrocarbon product preferably comprises alkanes, more preferably alkanes having from 5 to 100 carbon atoms, or higher. Such a hydrocarbon product may be particularly desirable. In addition, cobalt catalysts typically employed to produce such a product mixture may be particularly vulnerable to poisoning with hydrogen cyanide.

The co-produced water is preferably recovered from the Fischer-Tropsch reaction chamber. As noted above, in the Fischer-Tropsch reaction, hydrogen and carbon monoxide are converted to hydrocarbons and water. The use of a by-product water stream, rather than having to use another source of water such as boiler feedwater or demineralised water, simplifies the method and reduces the operating cost. In addition, because the co-produced water is recovered from the Fischer-Tropsch reaction chamber, the water is free of poisons. Therefore, the risk of introducing poisons that might deactivate the catalyst is reduced over comparable processes using other sources of scrubbing liquid. In addition, carbon dioxide contained in the third syngas, as well as carbon dioxide generated by side reactions in the Fischer-Tropsch reaction chamber, typically becomes dissolved in the co-produced water and may enhance the ammonia scrubbing capacity of the scrubbing liquid.

The method may further comprise recovering gas from the Fischer-Tropsch reaction chamber, which may be re-circulated to the Fischer-Tropsch reaction chamber. Such a gas may comprise, for example, carbon monoxide, carbon dioxide and/or hydrogen. Re-circulating gas comprising carbon monoxide and/or hydrogen may increase the efficiency of the process and improve the yield. Since carbon dioxide is inert, re-circulating gas comprising carbon dioxide may increase the concentration of carbon dioxide in the gas passing through the Fischer-Tropsch reaction chamber, i.e. to greater than 0.5% by volume, typically around 5% by volume. This may result in the co-produced water generated in the Fischer-Tropsch reaction chamber becoming saturated with carbon dioxide. As a result, when the carbon dioxide-enriched water is recovered from the Fischer-Tropsch reaction chamber, the removal efficiency of ammonia may be increased. Use of water recovered from a hydrocarbon synthesis reaction chamber as a scrubbing liquid is described, for example, in U.S. Pat. No. 6,107,353 (see discussion above). However, in U.S. Pat. No. 6,107,353 the recovered water is first stripped with natural gas to remove organic acids and oxygenates so that they do not contaminate the final hydrogen-cyanide/ammonia absorption zones. Such stripping also removes carbon dioxide. In contrast, in the method of the present invention there is no need to carry out stripping of the water prior to use as a scrubbing liquid. Furthermore, the method of the present invention is capable of reducing the hydrogen cyanide and ammonia in the syngas to ppb levels without the use of downstream hydrogen-cyanide/ammonia absorption zones between the scrubbing stage and the Fischer-Tropsch reaction chamber.

Providing a first syngas containing hydrogen cyanide preferably comprises the gasification of biomass and/or municipal waste. Biomass and municipal waste are low cost and becoming more widely available, and syngas produced from these species may be particularly suitable for the production of liquid hydrocarbons. Gasification is a technique known in the art. During gasification, the biomass and/or municipal waste is blown through with oxygen and steam (water vapour) while also being heated (and in some cases pressurized). It is essential that the oxidizer supplied is insufficient for complete oxidation (combustion) of the fuel. During the reactions mentioned, oxygen and water molecules oxidize the biomass and/or municipal waste and produce a gaseous mixture of carbon dioxide, carbon monoxide, water vapour, and molecular hydrogen.

Advantageously, heat may be recovered from the gasification for use in other steps of the method. Alternatively, providing a first syngas containing hydrogen cyanide is preferably carried out by the steam reforming of a hydrocarbon feedstock or recovering the syngas from a reverse-water-gas-shift unit. Syngas produced from these species may be particularly suitable for the production of liquid hydrocarbons.

Preferably, the first syngas has been subjected to a step of carbon dioxide removal. This may be achieved using conventional carbon dioxide removal apparatus, such as an acid gas removal unit (AGRU). The carbon dioxide removal preferably operates by means of absorption using a suitable liquid that removes the carbon dioxide from the first syngas. This may make the method more efficient, since a reduced volume of inert gas will reduce the energy required to carry out any heating or cooling steps.

The first syngas may further comprise ammonia. The method of the present invention is particularly suitable for use on a syngas containing ammonia in view of the high ammonia removal capacity of the scrubbing liquid.

The first syngas may further comprise sulphur-containing gas. In this case, the method preferably further comprises removing at least some of the sulphur-containing gas from the syngas prior to the step of converting at least a portion of the hydrogen cyanide to ammonia. Removing at least some of the sulphur-containing gas from the syngas preferably comprises contacting the syngas with a solvent at a pressure of at least 1 MPa to dissolve at least some of the sulphur-containing gas. Removing at least some of the sulphur-containing gas from the syngas preferably comprises contacting the syngas with a suitable sulphur absorbent. This may usefully be achieved at the same time as carbon dioxide removal using an acid gas removal unit (AGRU).

Converting at least a portion of the hydrogen cyanide to ammonia to provide the second syngas preferably comprises catalytic hydrolysis of the hydrogen cyanide. Catalytic hydrolysis is a simple way of converting hydrogen cyanide to ammonia since it may be carried out by the addition of water and at relatively low temperatures. Accordingly, the simplicity and efficiency of the method may be improved. The water for the hydrolysis may be either sprayed into the syngas stream before it is fed to the hydrolysis bed or added to the syngas as steam. The equilibrium reaction for this is:

HCN+H₂O [symbol] NH₃+CO

The hydrolysis is preferably carried out at a temperature of greater than 100° C., more preferably from 150° C. to 300° C. Lower temperatures may result in unfavourably low levels of hydrolysis. Higher temperatures may increase the energy cost of the method without a significant improvement in hydrogen cyanide conversion.

The hydrolysis is preferably carried out using an alumina catalyst, more preferably an activated alumina catalyst. Such catalysts may result in a particularly high conversion rate and/or enable operation at a favourably low temperature.

Following hydrolysis, the second syngas is preferably cooled prior to contacting the scrubber, more preferably to a temperature of 40° C. or less, even more preferably to ambient temperature.

The second syngas preferably comprises less than 10 ppbv hydrogen cyanide. Such a low level of hydrogen cyanide may result in a particularly low level of poisoning of the Fischer-Tropsch catalyst.

The third syngas preferably comprises less than 10 ppbv ammonia. Such a low level of hydrogen cyanide may result in a particularly low level of poisoning of the Fischer-Tropsch catalyst.

Despite upstream treatment of the first syngas, it is possible that the second syngas will contain traces of sulphur compounds, which are desirably removed. Preferably, the method further comprises passing the second syngas to a sulphur guard bed to remove sulphur compounds from the second syngas prior to passing the second syngas to the scrubber. The sulphur guard bed preferably comprises a zinc-oxide catalyst. Removal of residual sulphur-containing species from the second syngas may reduce poisoning of the Fischer-Tropsch catalyst by sulphur.

The temperature of the Fischer-Tropsch reaction chamber is preferably from 150° C. to 300° C. Lower temperatures may result in unfavourably low levels of liquid hydrocarbons being generated. Higher temperatures may increase the energy cost of the method without a significant increase in the levels of liquid hydrocarbons being produced.

Passing the third syngas through a Fischer-Tropsch reaction chamber to produce a liquid hydrocarbon product comprises contacting the third syngas with a catalyst comprising a metal selected from cobalt, iron and ruthenium, preferably cobalt. Such a catalyst may be particularly effective at catalysing Fischer-Tropsch reactions and/or enable the reaction to proceed at favourably low temperatures and/or with high yield.

The hydrocarbon products from the Fischer Tropsch reaction chamber are separated from co-produced water and unreacted gases, and then may be converted, for example by hydrocracking, into liquid hydrocarbon fuels.

The invention will now be described in relation to the following non-limiting examples.

### EXAMPLE

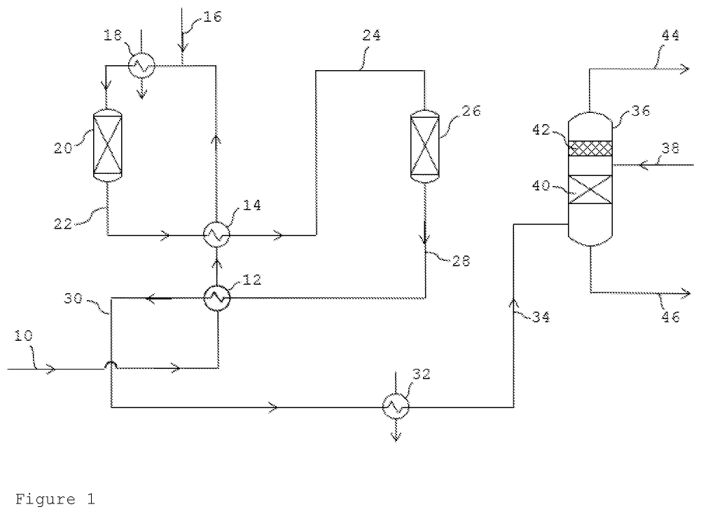

FIG. 1 shows a flow diagram of an example of a method according to the present invention. In the example method, a first syngas is generated by gasification of biomass (not shown). The first syngas then undergoes acid-gas removal (not shown). After acid-gas removal, the first syngas 10 is heated in a Syngas Polishing Interchanger 12 using hot gas from a downstream Sulphur Guard Bed, 26, to around 125° C. Following this, the syngas passes through another Syngas Polishing Interchanger 14, where it is heated to around 190° C. using the gas from hydrogen-cyanide Hydrolysis Bed 20. The syngas is further heated to 250° C. by the Hydrogen Cyanide Hydrolysis Bed Preheater, 18, using Saturated high-pressure Steam. There is a boiler feed water supply 18 upstream of Hydrogen Cyanide Hydrolysis Bed 20 to provide sufficient water for hydrolysis of COS and HCN. After cooling of the second syngas 22 to 150° C. through Syngas Polishing Interchanger 14, the cooled second syngas 24 enters a final clean-up Sulphur Guard Bed 26, which contains a zinc-oxide catalyst. The desulphurised second syngas 28 is then cooled in Syngas Polishing Interchanger 12 before subsequent cooling of the syngas 30 in the Syngas Wash Drum Cooler, 32 to 40° C. Ammonia, either present in the gas or formed by hydrogen-cyanide hydrolysis, is washed from the syngas 34 in the Syngas Wash Drum, 36 using FT co-produced water 38 from a downstream FT Unit (not shown). The syngas wash water 46 from the Syngas Wash Drum bottoms is sent for treatment. The purified third syngas is recovered from the Syngas Wash Drum 36 via line 44 and send directly to the FT Unit (not shown). The Syngas Wash Drum 36 contains either trays, structured packing or random packing 40 to provide mass-transfer surface area and a demister 42 to prevent liquid carry over. The water wash rate and number of mass-transfer stages is set to achieve the required ammonia removal level i.e. <10 ppbv.

The foregoing detailed description has been provided by way of explanation and illustration and is not intended to limit the scope of the appended claims. Many variations in the presently preferred embodiments illustrated herein will be apparent to one of ordinary skill in the art and remain within the scope of the appended claims and their equivalents.

The method depicted in FIG. 1 was modelled using standard process software. The conditions and compositions of the streams were as follows:

| Stream No. | | 10 | 16 | 22 | 28 | 38 | 44 | 46 |
|---|---|---|---|---|---|---|---|---|
| Temperature | ° C. | 40 | 105 | 250 | 150 | 40 | 40 | 40 |
| Pressure | bara | 40.0 | 39.5 | 39.1 | 38.6 | 38.1 | 37.9 | 38.1 |
| Flow | Kmol/h | 100.0 | 2.0 | 102.0 | 102.0 | 6.2 | 100.2 | 8.0 |
| Composition | | | | | | | | |
| Water | mol % | 0.00 | 100.00 | 1.96 | 1.96 | 99.30 | 0.20 | 99.80 |
| Hydrogen | mol % | 66.38 | 0.00 | 65.08 | 65.08 | 0.00 | 66.23 | 0.00 |
| Carbon | mol % | 31.61 | 0.00 | 30.99 | 30.99 | 0.00 | 31.54 | 0.00 |
| Monoxide | | | | | | | | |
| Carbon | mol % | 0.50 | 0.00 | 0.49 | 0.49 | 0.10 | 0.50 | 0.00 |
| Dioxide | | | | | | | | |
| Methane | mol % | 0.50 | 0.00 | 0.49 | 0.49 | 0.00 | 0.50 | 0.00 |
| Methanol | mol % | 0.01 | 0.00 | 0.01 | 0.01 | 0.60 | 0.03 | 0.20 |
| Nitrogen | mol % | 0.50 | 0.00 | 0.49 | 0.49 | 0.00 | 0.50 | 0.00 |
| Argon | mol % | 0.50 | 0.00 | 0.49 | 0.49 | 0.00 | 0.50 | 0.00 |
| H₂S | ppb(molar) | 50 | 0 | 93 | 5 | 0 | 5 | 0 |
| COS | ppb(molar) | 50 | 0 | 5 | 5 | 0 | 5 | 0 |
| HCN | ppb(molar) | 100 | 0 | 5 | 5 | 0 | 5 | 6 |
| NH₃ | ppb(molar) | 1000 | 0 | 1073 | 1073 | 0 | 10 | 13584 |

1. A method of producing liquid hydrocarbons from a syngas, the method comprising: providing a first syngas containing hydrogen cyanide;
converting at least a portion of the hydrogen cyanide in the first syngas to ammonia to provide a second syngas enriched in ammonia and depleted in hydrogen cyanide;
passing the second syngas to a scrubber and contacting the second syngas with a scrubbing liquid, whereby at least a portion of ammonia contained in the second syngas is retained in the scrubbing liquid to form a third syngas depleted in ammonia and hydrogen cyanide; and
passing the third syngas through a Fischer-Tropsch reaction chamber to produce a liquid hydrocarbon product, wherein passing the third syngas through a Fischer-Tropsch reaction chamber to produce a liquid hydrocarbon product comprises contacting the third syngas with a catalyst comprising a metal selected from cobalt, iron and ruthenium; wherein the scrubbing liquid comprises a coproduced water separated from products recovered from the Fischer-Tropsch reaction chamber.

2. The method of claim 1, wherein the co-produced water comprises at least 0.01 mol/L carbon dioxide, preferably at least 0.02 mol/L carbon dioxide.

3. The method of claim 1, wherein the co-produced water is saturated with carbon dioxide under the temperature and pressure conditions of the scrubber.

4. The method of claim 1, wherein the liquid hydrocarbon product comprises alkanes.

5. The method of claim 1, wherein providing a first syngas containing hydrogen cyanide, comprises the gasification of biomass and/or municipal waste, or by the steam reforming of a hydrocarbon feedstock or wherein the first syngas is recovered from a reverse-water-gas shift unit.

6. The method of claim 1, wherein the first syngas has been subjected to a step of carbon-dioxide removal.

7. The method of claim 1, wherein the first syngas further comprises ammonia.

8. The method of claim 1, wherein converting at least a portion of the hydrogen cyanide to ammonia to provide the second syngas comprises catalytic hydrolysis of the hydrogen cyanide.

9. The method of claim 8, wherein the hydrolysis is carried out at a temperature of greater than 100° C., preferably from 150° C. to 300° C.

10. The method of claim 8, wherein the hydrolysis is carried out using an alumina catalyst, preferably an activated alumina catalyst.

11. The method of claim 1, wherein the second syngas comprises less than 10 ppbv hydrogen cyanide.

12. The method of claim 1, wherein the third syngas comprises less than 10 ppbv ammonia.

13. The method of claim 1, wherein the method further comprises passing the second syngas to a sulphur guard bed to remove sulphur compounds from the second syngas prior to passing the second syngas to the scrubber.

14. The method of claim 1, wherein the temperature of the Fischer-Tropsch reaction chamber is from 150° C. to 300° C.

15. The method of claim 1, wherein passing the third syngas through a Fischer-Tropsch reaction chamber to produce a liquid hydrocarbon product comprises contacting the third syngas with a catalyst comprising cobalt.
